(19) United States
(12) Patent Application Publication
(10) Pub. No.: US 2024/0139013 A1
(43) Pub. Date: May 2, 2024

(54) WRIST COMPRESSION DEVICE FOR CARPAL ARCH SPACE AUGMENTATION

(71) Applicant: Arizona Board of Regents on Behalf of the University of Arizona, Tucson, AZ (US)

(72) Inventor: Zong-Ming Li, Tucson, AZ (US)

(21) Appl. No.: 18/493,245

(22) Filed: Oct. 24, 2023

Related U.S. Application Data
(60) Provisional application No. 63/421,938, filed on Nov. 2, 2022.

(51) Int. Cl. A61F 5/01

(52) U.S. Cl. CPC A61F 5/0118; A61F 5/012

(57) ABSTRACT

A wrist device, system, or method for compressing a wrist that includes a shell, a compression mechanism, and an actuator. The shell is shaped to conform to the wrist, has a radius side and an ulna side opposite the radius side, and a thumb support. The compression mechanism is associated with the radius and ulna sides of the shell. The actuator is associated with the shell and is configured to provide compression in a radioulnar direction to opposing sides of the wrist via the compression mechanism.

### CROSS-REFERENCE TO RELATED APPLICATION

This application claims the benefit of U.S. Provisional Application No. 63/421,938, filed on Nov. 2, 2022, entitled Wrist Compression Device for Carpal Arch Space Augmentation, the subject matter of which is incorporated by reference in its entirety.

### BACKGROUND

The present disclosure relates generally to carpal arch space augmentation of the wrist and devices configured to treat carpel tunnel syndrome via radioulnar wrist compression to alleviate median nerve compression at the wrist.

Carpal tunnel syndrome (CTS) is caused by compression of the median nerve at the wrist, resulting in symptoms of pain and paresthesia. If left untreated, irreversible degradation of the median nerve can occur, leading to hand dysfunction. As the most common compressive neuropathy, CTS affects 3-5% of the US general population. Women and individuals who perform repetitive hand and wrist motions are among those most commonly affected by the disorder. CTS substantially impacts worker productivity, activities of daily life and the patient's well-being.

Despite the widespread burden of this disease, clinical management of CTS can be challenging for both physicians and patients. Current treatment options fall into either conservative or surgical intervention categories. Non-invasive methods to alleviate CTS symptoms include nonsteroidal anti-inflammatory drugs, corticosteroids, stretching, splinting, massage, and laser treatment; however, the effectiveness of those modalities remains inadequate and inconclusive. Carpal tunnel release surgery to transect the transverse carpal ligament is another solution to decompress the median nerve. Unfortunately, the surgical procedure unavoidably disrupts essential anatomical, biomechanical and physiological functions of the wrist and is often associated with postoperative complications, such as reduced grip strength, pillar pain, carpal bone instability, scar tissue formation, and perineural fibrosis.

### SUMMARY

In order to decompress the median nerve of the wrist non-surgically, the present disclosure provides a biomechanical approach to augment the carpal arch area. When an external force is applied and directed across the width of the wrist in the radioulnar direction, the distance between the trapezium and hook of hamate narrows, bowing the transverse carpal ligament palmarly. The mechanisms of wrist compression for carpal arch space augmentation and its implications to decompress the median nerve. In particular, radioulnar wrist compression can alleviate median nerve compression and restore impaired neurophysiological and biomechanical functions of the nerve in CTS patients, as indicated by improvements in median nerve flattening, nerve mobility, cross sectional area, and distal motor latency.

A wrist device of the present disclosure for compressing a wrist may comprise a shell shaped to conform to the wrist and provide thumb support, the shell having a radius side and an ulna side opposite the radius side and an actuator associated with the shell which is configured to achieve carpal arch space augmentation by providing compression in the radioulnar direction to the radius and ulna sides of the wrist.

A wrist device for compressing a wrist of the present disclosure may comprise a shell shaped to conform to the wrist and provide a thumb support, wherein the shell has a radius side and an ulna side opposite the radius side. A compression mechanism is associated with the radius and ulna sides of the shell. An actuator is associated with the shell and is configured to provide compression in the radioulnar direction to opposing sides of the wrist via the compression mechanism.

In certain embodiments, the shell comprises two rigid parts including a radius side and an ulna side; the shell is a rigid one-piece shell and the thumb support is a hollow extension of the shell; the compression mechanism is at least one compression pad positioned on an inside of the shell at each of the radius and ulna sides thereof; the compression mechanism is at least one air balloon positioned on an inside of the shell at each of the radius and ulna sides thereof; and/or the actuator is an adjustable pneumatic mechanism; and/or the adjustable pneumatic mechanism is one or more pumps.

In other embodiments, the shell comprises two rigid separable parts, one including the radius side and the other including the ulna side; the radius side incorporates the thumb support; the actuator is an adjustable torque mechanism coupled to a top surface of the shell; the adjustable torque mechanism includes cooperating upper and lower slides, and one of the upper and lower slides is coupled to the radius side and the other of the upper and lower slides is coupled to the ulna side; the adjustable torque mechanism includes a translation mechanism coupled to the upper and lower slides, the translation mechanism being configured to slide the upper and lower slides in and out with respect to one another along the radioulnar direction; the translation mechanism is a fastener; and/or the translation mechanism includes a torque limiting feature configured to limit the compression in the radioulnar direction.

In an embodiment, the shell can be a glove or wrist cuff. In another embodiment, an elastic band or spring is the compression mechanism and the actuator.

A wrist device for compressing a wrist may comprise a shell shaped to conform to the wrist, wherein the shell has a radius side and an ulna side opposite the radius side. A compression mechanism is associated with the radius and ulna sides of the shell. An actuator is associated with the shell and is configured to provide radioulnar compression to the radius and ulna sides of the wrist via the compression mechanism. A control unit is operatively connected to the compression mechanism and the actuator and is programmed to control the operation of the compression mechanism.

In some embodiments, the compression mechanism is at least one air balloon positioned inside of the shell at the radius and ulna sides; the control unit includes a display with a user interface configured to allow user input to control operation of the compression mechanism; and/or the control unit is integrated to a wearable device.

This summary is not intended to identify essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

### BRIEF DESCRIPTION OF THE FIGURES

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIGS. 1A and 1B are perspective and exploded views, respectively, of an exemplary device, according to one embodiment;

FIGS. 2A and 2B are perspective and exploded views, respectively, of an exemplary device, according to another embodiment;

FIGS. 3A and 3B are perspective and exploded views, respectively, of an exemplary device, according to yet another embodiment; and

FIG. 4 is a view of an exemplary device of still another embodiment.

### DETAILED DESCRIPTION

The present disclosure relates to devices, systems, and methods designed to apply pressure to both sides of the wrist in the radioulnar direction (also referred to as radioulnar wrist compression) to treat CTS. The devices are configured to apply radioulnar wrist compression to alleviate sensory symptoms, such as tingling and numbness, attributable to CTS. The radioulnar wrist compression provided by the device can also improve hand motor impairment, such as pinch and grasp weakness.

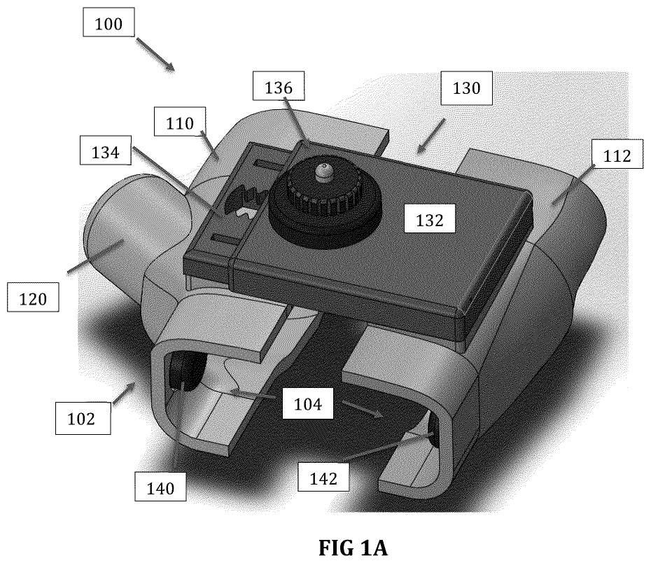

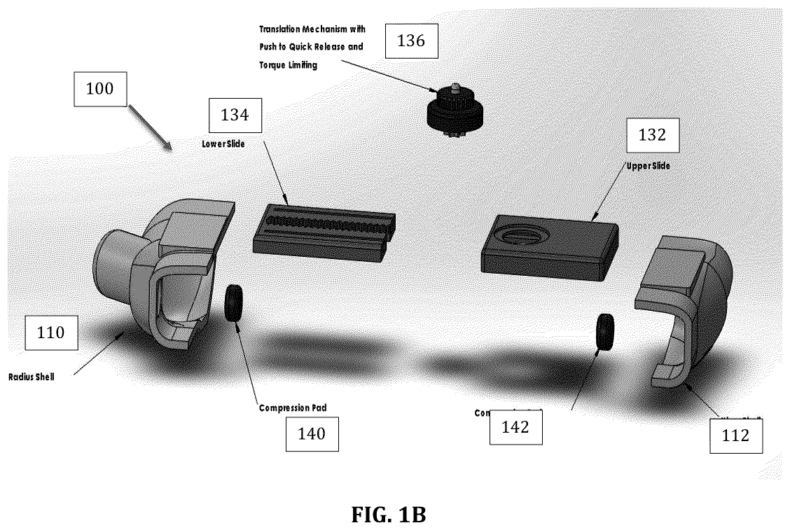

FIGS. 1A and 1B illustrate one embodiment of a device 100 of the present disclosure in which torque or torque system is used to apply the pressure to both sides of the wrist by adjusting a translation mechanism. The device 100 may comprise a shell 102 formed of two cooperating parts, a radius side or part 110 and an ulnar side or part 112, where the radius part 110 is shaped to cover the thumb side of the wrist and the ulnar part is shaped to cover the ulnar side of the wrist that is opposite the thumb side. The radius part 110 has a hollow thumb extension 120 that receives the user's thumb. Although FIGS. 1A and 1B show the device 100 for the right hand wrist, the device 100 can be configured for use on either wrist.

The device 100 has an actuator 130 that can be a torque member that fits on top portions of the shell parts 110 and 112. The torque member 130 is configured to pull the two parts 110 and 112 of the shell 102 together in the radioulnar direction, that is the direction relating to the radius and ulna bones of the wrist, to apply the radioulnar compression. In an example, the torque member 130 may comprise upper and lower slides 132 and 134 that cooperate with a translation mechanism 136 that allows the upper and lower slides 132 and 134 to slide in and out with respect to another along the radioulnar direction. The slides 132 and 134 are nested and slide past each other during adjustment. The slides 132 and 134 may prevent any foreign object from interfering with torque member's operation. The slides 132 and 134 may be formed of a material such as, common plastic, like ABS etc.

The translation mechanism 136 can be a fastener, such as a screw. In an example, the translation member has a right angle adjustment knob and two guide rails to maintain the three dimensional orientation of the two shell parts 110 and 120. The translation mechanism 136 may have a torque limiting feature designed to prevent overtightening of the shell parts 110 and 112, that is limit the maximum pressure that can be applied to the wrist. The translation mechanism 136 can incorporate a quick release feature configured to release the engagement of the upper and lower slides 132 and 134 to allow for easy removal of the device from the user's hand.

A compression mechanism 104 is associated with the shell 102. The compression mechanism 104 may be, for example, first and second compression pads 140 and 142 that can be provided inside each of the shell parts 110 and 112 to apply pressure to the appropriate locations on the radius and ulna sides, respectively, of the wrist to provide the radioulnar compression to the wrist. A pocket can be provided on the inside of each shell part 110 and 112 to hold the first and second compression pads, respectively. More than one of the first and second pads 140 and 142 can be used with the shell parts 110 and 112. The pads 140 and 142 can be made of any compressible material, such as silicone or some other soft, compliant material.

In an alternative embodiment, the wrist compression provided by the device in the radioulnar direction can be achieved by an electromagnet. For example, the lower slide 134 can be attached to an electromagnet that pulls the upper slide 132 to move the slides 132 and 134 together. The electromagnet can be calibrated to generate a desired force in the radioulnar direction through the compression pads 140 and 142. The electromagnet can be connected to a control that is programmed for needed compression duration and on/off cycle time.

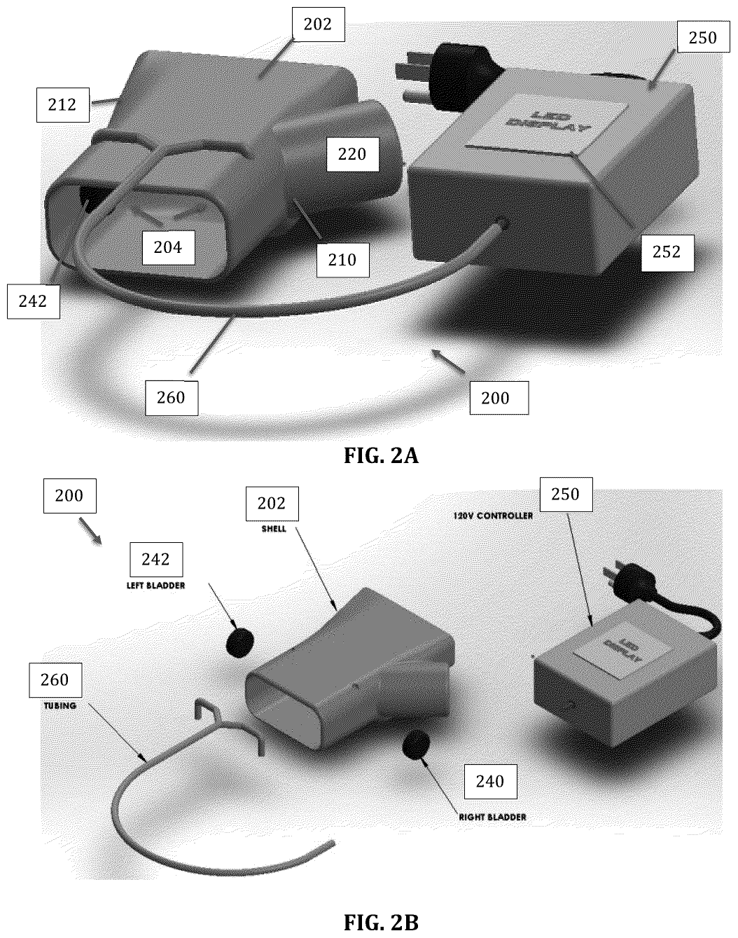

FIGS. 2A and 2B illustrate another embodiment of a device 200 of the present disclosure in which a pneumatic system is used to apply the wrist pressure. The radial and ulnar shell parts of the above embodiment are essentially combined into one rigid shell structure. The shell may be a thin, rigid device made of a hard plastic or other suitable material that will contain the hand of the user and provide separate support for the thumb.

The compression mechanism is implemented by air balloons (in lieu of compression pads). The balloons are inflated and deflated by electronically turn on/off micro air pumpers. The compression force, on/off frequency, and duration are adjustable on a monitor, such as a touchscreen LED/LCD monitor. The electronic control unit can be integrated to a wearable device like the portable wrist cuff. Alternatively, the control unit through which an air conduction tube is connected to the air balloons in shell is external to the shell.

In an example, the compression mechanism 204 can be air balloons 240 and 242, instead of the compression pads of the above embodiment, to apply the radioulnar compression to the wrist. The device 200 may comprise a shell 202 that is one-piece with a radius side or part 210 and an ulnar side 212 where the radius side 210 is shaped to cover the thumb side of the wrist and the ulnar side 212 is shaped to cover the ulnar side of the wrist that is opposite the thumb side. The radius part 210 has a hollow thumb extension 220 that receives the user's thumb. Although FIGS. 2A and 2B show the device 200 for the left hand wrist, the device 200 can be configured for use on either wrist.

The balloons 240 and 242 can be inflated and deflated to the appropriate pressure for applying the radioulnar compression. Each balloon 240 and 242 may be a bladder formed of an elastic material, such as rubber. The balloons 240 and 242 can be connected to an actuator, such micro air pumpers or pumps, for example, that can be operated by a control unit 250. The balloons 240 and 242 can be connected to the control or electronic unit 250, which is programmed to control and adjust the pressure in the balloons 240 and 242 via the pumps as needed to control and adjust the compression force, the on/off frequency, and the duration of the compression. A display 252, such as an LCD or LED monitor or the like, has a user interface, such as a touch screen, can be connected to or part of the control 250 and allows the user to input and control the operation of the balloons 240 and 242.

In an example, the balloons 240 and 242 can be rubber bladders that can be attached to the shell and connected to the controller or control unit 250 via flexible tubing 260. The pressurization of the bladders will provide the prescribed pressure to both sides of the wrist. The controller 250 may comprise of a microcontroller programmed to the system, such as to turn the pressure on and off via the pumpers and adjust the pressure to the balloons as needed. The display is connected to the control 250 and allows the user to interface with the system. Power can be provided to the device via a rechargeable battery, a power cord that will connect to any standard electric plug, or the like.

The device 200 and system can be operated via the display 252 to initiate a treatment sequence. For example, the treatment sequence may comprise automatically applying a pressurization of the bladders for a set period of time, and then a release of pressure for a separate period of time in a cyclical manner. The amount of force applied to the wrist can be accurately controlled via the control unit 250 by the amount of pressure in the bladders created by the pumps. The time intervals can be altered by inputs to the microcontroller. The device can use rechargeable batteries for the powering the operation allowing the unit to be portable. The device may have a power cord that will connect to any standard electric plug.

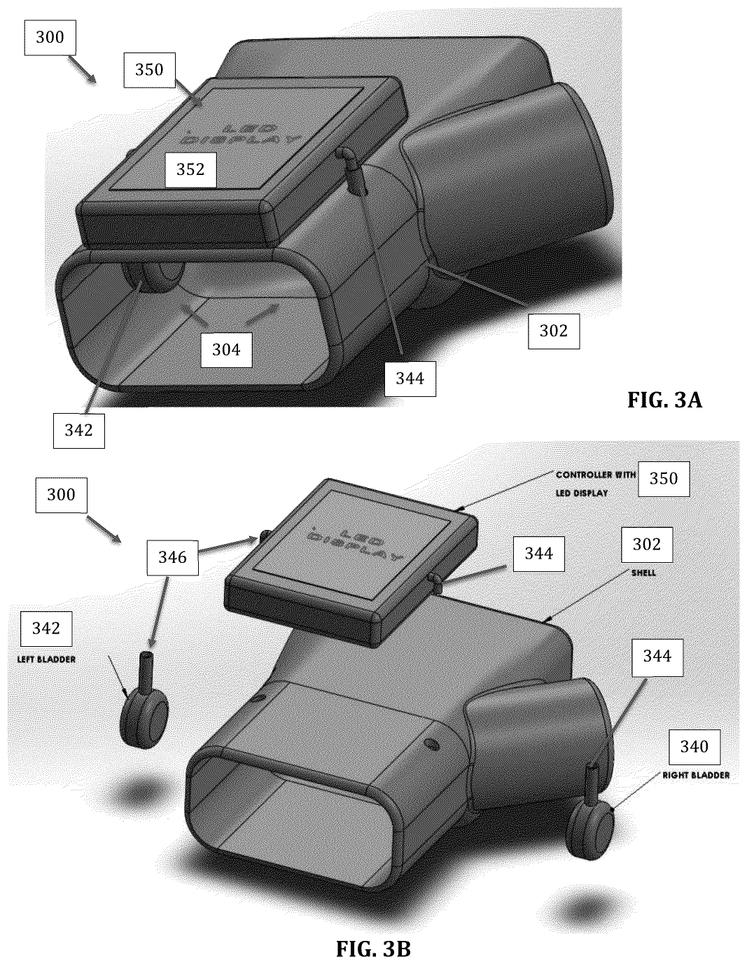

FIGS. 3A and 3B illustrate still another exemplary device 300 of the present disclosure which is similar to the device 200 of the above embodiment, except that the control unit 350 and its display 352 are attached the directly to the shell 302. The control unit 350 can reside on the top of the shell 302 and can be connected to the compression mechanism 304, such as air balloons 340 and 342, via respective tubes 344 and 346 that extend through the holes in the top of the shell 302 to connect with each balloon 340 and 342. The control unit 350 may also incorporate a rechargeable battery and/or have a power cord.

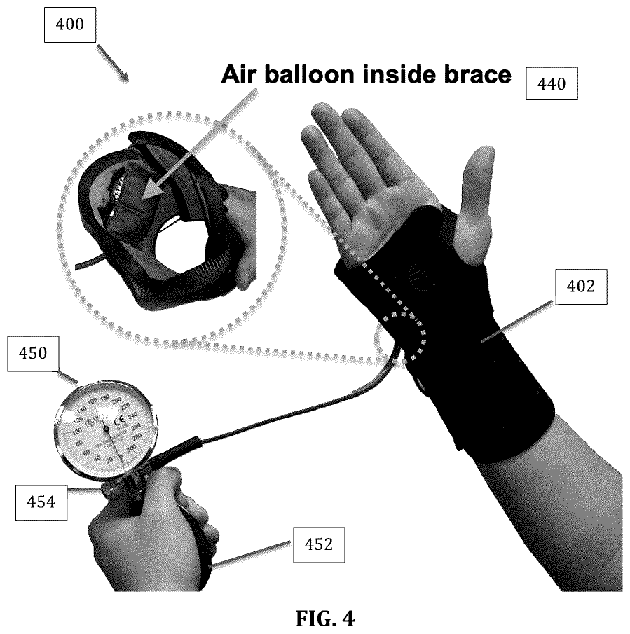

FIG. 4 illustrates another exemplary embodiment of the present disclosure that is a device 400 that is wearable brace, glove, cuff, or the like 402 with an inflatable balloon bladder 440 inside the brace for radioulnar wrist compression. To facilitate self-use, the device 400 is configured as a portable, wearable wrist device that produces the biomechanical specifications of the disclosure in the radioulnar direction. This device 400 may have a thermoformable Exos Wrist Brace with Boa (DJO, Vista, CA), capable of molding to the patient's wrist for a custom fit in an anatomically neutral position.

The air bladder 440 (e.g. 50 mm×50 mm bladder) can be attached to the inside of the brace and centered around the hamate level on the medial side of the wrist. To inflate the bladder 440 and apply wrist compression in the radioulnar direction, a dial 450, such as an aneroid sphygmomanometer dial, a bulb 452, and air valve 454 can be attached to the bladder 440, such as via a hose, and cooperate with one another to provide air pressure to the bladder 440 by squeezing the bulb 452.

In embodiments, the wrist compression in the radioulnar direction to achieve the carpal arch space augmentation can be realized by elastic compression, such by use of one or more elastic bands or springs. In an example, a fingerless glove can be equipped with the elastic band. The palmer wrist area is made of an archable plate with its ends naturally curved around the radial and ulnar sides. Two compression pads can be placed inside the radial and ulnar ends of the glove aligning with the trapezium and hamate. The elastic band can be wrapped around the wrist to generate compression force in the radioulnar direction through the compression pads while the palmer side is protected from archable plate. The springs can be connected to the radius and ulna shells to generate compressive force when the springs are stretched.

CTS patients can be trained to perform a wrist compression intervention protocol or treatment sequence and/or instructions provided for at-home reference use for using a device and system of the present disclosure. An exemplary treatment protocol may be a four-week intervention period, during which patients can perform three radioulnar wrist compression sessions daily using a device of the present disclosure. Each session can include three 5-minute wrist compression with a 1-minute rest between intervals, for example. A 10 N compressive force can be achieved by inflating the balloon or bladder to a pre-calibrated pressure of 140 mmHg. In the above embodiments, the control unit can be programmed to optimize the force application dosage (magnitude, duration, and frequency) for different severity of CTS.

Using the devices and system of the present disclosure in a treatment sequence, users, e.g. patients, can experience relief from sensory symptoms, including pain, numbness, tingling and dermal sensation, over functional symptoms due to the fact that sensory nerve fibers are predominantly affected in CTS. The most characteristic manifestation of CTS is the sensation of numbness and tingling (paresthesia). During four weeks of daily radioulnar wrist compression, patients may find that symptoms of paresthesia are relieved. And by the second week of the treatment, intervention symptoms are improved, and remained stable throughout the remaining two weeks of intervention such that no significant differences in pain were observed between weeks 2 and 4.

It will be apparent to those skilled in the art having the benefit of the teachings presented in the foregoing descriptions and the associated drawings that modifications, combinations, sub-combinations, and variations can be made without departing from the spirit or scope of this disclosure. Likewise, the various examples described may be used individually or in combination with other examples. Those skilled in the art will appreciate various combinations of examples not specifically described or illustrated herein that are still within the scope of this disclosure. In this respect, it is to be understood that the disclosure is not limited to the specific examples set forth and the examples of the disclosure are intended to be illustrative, not limiting.

As used in this specification and the appended claims, the singular forms “a”, “an” and “the” include plural referents, unless the context clearly dictates otherwise. Similarly, the adjective “another,” when used to introduce an element, is intended to mean one or more elements. The terms “comprising,” “including,” “having” and similar terms are intended to be inclusive such that there may be additional elements other than the listed elements.

Additionally, where a method described above or a method claim below does not explicitly require an order to be followed by its steps or an order is otherwise not required based on the description or claim language, it is not intended that any particular order be inferred. Likewise, where a method claim below does not explicitly recite a step mentioned in the description above, it should not be assumed that the step is required by the claim.

It is noted that the description and claims may use geometric or relational terms, such as, such as right, left, above, below, upper, lower, top, bottom, linear, arcuate, elongated, parallel, perpendicular, etc. These terms are not intended to limit the disclosure and, in general, are used for convenience to facilitate the description based on the examples shown in the figures. In addition, the geometric or relational terms may not be exact. For instance, walls may not be exactly perpendicular or parallel to one another because of, for example, roughness of surfaces, tolerances allowed in manufacturing, etc., but may still be considered to be perpendicular or parallel.

What is claimed is:

1. A wrist device for compressing a wrist, comprising:
a shell shaped to conform to the wrist, the shell having a radius side and an ulna side opposite the radius side, and a thumb support;
a compression mechanism associated with the radius and ulna sides of the shell; and
an actuator associated with the shell, the actuator being configured to provide compression in a radioulnar direction to opposing sides of the wrist via the compression mechanism.

2. The device of claim 1, wherein the shell comprises two rigid separable parts, one including the radius side and the other including the ulna side.

3. The device of claim 2, wherein the radius side incorporates the thumb support.

4. The device of claim 1, wherein the shell is a rigid one-piece shell and the thumb support is a hollow extension of the shell.

5. The device of claim 1, wherein the compression mechanism is at least one compression pad positioned on an inside of the shell at each of the radius and ulna sides thereof.

6. The device of claim 1, wherein the actuator is an adjustable torque mechanism coupled to a top surface of the shell.

7. The device of claim 6, wherein the adjustable torque mechanism includes cooperating upper and lower slides, and one of the upper and lower slides is coupled to the radius side and the other of the upper and lower slides is coupled to the ulna side.

8. The device of claim 7, wherein the shell comprises two rigid separable parts, one including the radius side and the other including the ulna side.

9. The device of claim 7, wherein the adjustable torque mechanism includes a translation mechanism coupled to the upper and lower slides, the translation mechanism being configured to slide the upper and lower slides in and out with respect to one another along the radioulnar direction.

10. The device of claim 9, wherein the translation mechanism is a fastener.

11. The device of claim 9, wherein the translation mechanism includes a torque limiting feature configured to limit the compression in the radioulnar direction.

12. The device of claim 1, wherein the compression mechanism is at least one air balloon positioned on an inside of the shell at each of the radius and ulna sides thereof.

13. The device of claim 1, wherein the actuator is an adjustable pneumatic mechanism.

14. The device of claim 13, wherein the adjustable pneumatic mechanism is one or more pumps.

15. The device of claim 1, wherein the shell is a glove or wrist cuff.

16. The device of claim 15, wherein an elastic band or spring is the compression mechanism and the actuator.

17. A wrist device for compressing a wrist, comprising:
a shell shaped to conform to the wrist, the shell having a radius side and an ulna side opposite the radius side, and a thumb support;
a compression mechanism associated with the radius and ulna sides of the shell;
an actuator associated with the shell, the actuator being configured to provide radioulnar compression to the radius and ulna sides of the wrist via the compression mechanism; and
a control unit operatively connected to the compression mechanism and the actuator, the control unit being programmed to control the operation of the compression mechanism to provide the radioulnar compression.

18. The device of claim 17, wherein the compression mechanism is at least one air balloon positioned inside of the shell at the radius and ulna sides.

19. The device of claim 17, wherein the control unit includes a display with a user interface configured to allow user input to control operation of the compression mechanism.

20. The device of claim 17, wherein the control unit is integrated to a wearable device.

21. A method of wrist compression, comprising the step of using the wrist device of claim 1.

22. A method of wrist compression, comprising the step of using the wrist device of claim 17.
